(19) United States
(12) Patent Application Publication
(10) Pub. No.: US 2022/0218411 A1
(43) Pub. Date: Jul. 14, 2022

(54) DEVICES, SYSTEMS, AND METHODS FACILITATING NERVE ABLATION

(71) Applicant: Medtronic Holding Company Sárl, Tolochenaz (CH)

(72) Inventors: Calin Druma, San Jose, CA (US); Brian L. Ranck, Minneapolis, MN (US)

(21) Appl. No.: 17/546,194

(22) Filed: Dec. 9, 2021

Related U.S. Application Data
(60) Provisional application No. 63/135,075, filed on Jan. 8, 2021.

(51) Int. Cl. A61B 18/14

(52) U.S. Cl. CPC A61B 18/1492; A61B 2018/0044

(57) ABSTRACT

An ablation device includes a handle, an elongated body extending distally from the handle, and an end effector assembly selectively deployable relative to the elongated body. The end effector assembly includes a shaft having a curved configuration defining an inside portion and an outside portion. A plurality of electrode tines extends from the inside portion. At least one electrode tine and the shaft are configured to conduct RF energy therebetween and through tissue to treat tissue. Another end effector assembly includes a tongue having a concave side corresponding to an inside portion and a convex side corresponding to an outside portion. A plurality of electrodes is disposed on the concave side of the tongue and an insulating layer is disposed on the convex side of the tongue. At least one electrode and the tongue are configured to conduct RF energy therebetween and through tissue to treat tissue.

### CROSS-REFERENCE TO RELATED APPLICATIONS

This application claims the benefit of, and priority to, U.S. Provisional Patent Application No. 63/135,075, filed on Jan. 8, 2021, the entire contents of which are hereby incorporated herein by reference.

### FIELD

The present disclosure relates generally to surgical devices, systems, and methods and, more particularly, to devices, systems, and methods facilitating nerve ablation, e.g., posterior ablation of the basivertebral nerve.

### BACKGROUND

Back pain is a very common health problem. Low back pain, in particular, is a serious medical and social problem that is the most expensive occupational disorder in the United States and the leading cause of disability worldwide. Low back pain may be, for example, vertebrogenic, meaning the pain arises from problems within the vertebral bodies, or may be discogenic, meaning the pain arises from problems within the spinal discs. Vertebrogenic and discogenic pain may be caused by degenerative disease, metastases, and/or other conditions.

The basivertebral nerve of a vertebral body enters the posterior vertebral body via the basivertebral foramen and branches near the center of the vertebral body to innervate the superior and inferior endplates of the vertebral body. The basivertebral nerve has been found to transmit pain signals. Accordingly, ablating the basivertebral nerve is one potential avenue for alleviating vertebrogenic and/or discogenic low back pain.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an ablation device including a handle, an elongated body extending distally from the handle, and an end effector assembly selectively deployable relative to the elongated body. The end effector assembly includes a shaft and a plurality of electrode tines. The shaft has a curved configuration along at least a portion of a length thereof defining an inside portion and an outside portion. The shaft is adapted to connect to a source of Radio Frequency (RF) energy. The plurality of electrode tines is spaced-apart along the shaft and extends from the inside portion thereof. Each electrode tine of the plurality of electrode tines is adapted to connect to the source RF energy. At least one electrode tine of the plurality of electrode tines and the shaft are configured to conduct RF energy therebetween and through tissue to treat tissue.

In an aspect of the present disclosure, each electrode tine of the plurality of electrode tines includes a tissue-penetrating tip.

In another aspect of the present disclosure, at least two electrode tines of the plurality of electrode tines are configured to conduct RF energy therebetween.

In still another aspect of the present disclosure, the plurality of electrode tines is selectively extendable relative to the shaft.

In yet another aspect of the present disclosure, each electrode tine of the plurality of electrode tines defines a curved configuration and is curved in a similar direction as the shaft.

In still yet another aspect of the present disclosure, at least one cooling lumen extends through the shaft. The at least one cooling lumen is configured to receive cooling fluid to cool at least a portion of the shaft.

In another aspect of the present disclosure, the shaft is resiliently biased towards the curved configuration and is resiliently flexible from a substantially linear configuration, corresponding to a retracted position of the end effector assembly relative to the elongated body, to the curved configuration, corresponding to a deployed position of the end effector assembly relative to the elongated body.

Another ablation device provided in accordance with aspects of the present disclosure includes a handle, an elongated body extending distally from the handle, and an end effector assembly selectively deployable relative to the elongated body. The end effector assembly includes a tongue, a plurality of electrodes, and an insulating layer. The tongue has a curved configuration along at least a portion of a length thereof defining an inside portion and an outside portion. The tongue defines a curved transverse cross-sectional configuration defining a concave side and a convex side. The concave side corresponds to the inside portion and the convex side corresponds to the outside portion. The tongue is adapted to connect to a source of Radio Frequency (RF) energy. The plurality of electrodes is spaced-apart along the tongue and disposed on the concave side of the tongue. Each electrode of the plurality of electrodes is adapted to connect to the source RF energy. The insulating layer is disposed on the convex side of the tongue. At least one electrode of the plurality of electrodes and the tongue are configured to conduct RF energy therebetween and through tissue to treat tissue.

In an aspect of the present disclosure, at least two electrodes of the plurality of electrodes are configured to conduct RF energy therebetween.

In another aspect of the present disclosure, the tongue is resiliently biased towards the curved configuration and is resiliently flexible from a substantially linear configuration, corresponding to a retracted position of the end effector assembly relative to the elongated body, to the curved configuration, corresponding to a deployed position of the end effector assembly relative to the elongated body.

In still another aspect of the present disclosure, a user interface is disposed on the handle and includes a first control for selectively deploying the end effector assembly relative to the elongated body and a second control for selectively activating the supply of energy to the at least one electrode and the tongue.

In aspects of the present disclosure, the elongated body is resiliently flexible and/or defines a tissue-penetrating tip.

A method of ablating tissue provided in accordance with aspects of the present disclosure includes inserting an elongated body into a vertebral body of a patient anteriorly of a basivertebral nerve, deploying an end effector assembly from the elongated body such that a base electrode of the end effector assembly is positioned anteriorly of a basivertebral nerve with a plurality of electrodes of the end effector assembly facing posteriority towards the basivertebral nerve, and conducting RF energy between at least one electrode of the plurality of electrodes and the base electrode causing the RF energy to be conducted through the cancellous bone containing the basivertebral nerve to ablate the basivertebral nerve.

In an aspect of the present disclosure, deploying the end effector assembly orients the plurality of electrodes substantially radially inwardly towards the basivertebral nerve.

In another aspect of the present disclosure, deploying the end effector assembly includes penetrating the basivertebral nerve with at least one electrode of the plurality of electrodes.

In yet another aspect of the present disclosure, deploying the end effector assembly orients an insulating layer disposed on the base electrode on an anterior side of the base electrode to inhibit energy conduction anteriorly.

In still another aspect of the present disclosure, the method further includes circulating cooling fluid through a portion of the base electrode to promote energy conduction posteriorly.

In still yet another aspect of the present disclosure, conducting RF energy further includes conducting RF energy between at least two electrodes of the plurality of electrodes.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a side view of a surgical system provided in accordance with the present disclosure including an ablation device, an introducer, and a generator;

FIG. 2 is a side view of a distal portion of the ablation device of FIG. 1 with an end effector assembly thereof disposed in a deployed position;

FIG. 3 is a transverse, cross-sectional view of the end effector assembly of FIG. 2, taken across section line “3-3” of FIG. 2;

FIG. 4 is a transverse cross-section of a vertebral body including the end effector assembly of FIG. 2 positioned therein for ablating a basivertebral nerve;

FIG. 5 is a top view of a user interface of a handle of the ablation device of FIG. 1;

FIG. 6 is a front view of the generator of FIG. 1;

FIG. 7 is a side view of a distal end of the ablation device of FIG. 1 including another end effector assembly in accordance with the present disclosure, wherein the end effector assembly is disposed in a deployed position;

FIG. 8 is a transverse cross-section of a vertebral body including the end effector assembly of FIG. 7 positioned therein for ablating a basivertebral nerve; and

FIG. 9 is a schematic illustration of a robotic surgical system configured for use in accordance with the present disclosure.

### DETAILED DESCRIPTION

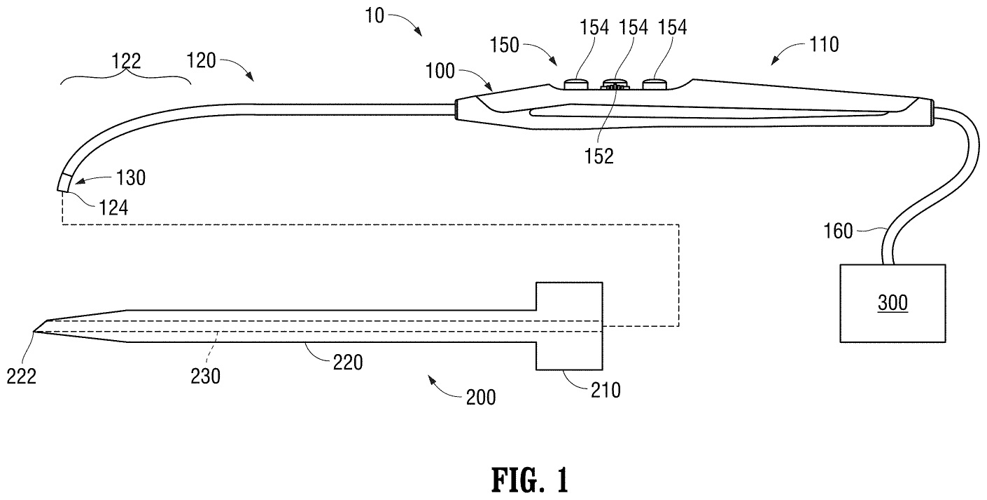

FIG. 1 illustrates a surgical system in accordance with the present disclosure shown generally identified by reference numeral 10. Surgical system 10 may be configured to facilitate nerve ablation, e.g., posterior ablation of the basivertebral nerve, as detailed below, although it is also contemplated that surgical system 10 be utilized to facilitate treatment of other tissue structures and/or at other anatomical locations. Surgical system 10 generally includes an ablation device 100, an introducer 200, and a generator 300. In some configurations, introducer 200 is omitted and ablation device 100 itself functions as the introducer. Additional or alternative components of system 10 are also contemplated such as, for example, a stylet, guidewire, trocar, etc.

Ablation device 100 includes a handle 110, an elongated body 120 extending distally from handle 110, an end effector assembly 130 selectively deployable from a distal end portion of elongated body 120, a user interface 150 disposed on handle 110, and a cable 160 extending proximally from handle 110 to enable connection of ablation device 100 with generator 300. Handle 110 is configured to facilitate grasping and manipulation by a user and, as noted above, includes user interface 150 disposed thereon to enable user-controlled actuation and activation of ablation device 100, as detailed below. Although handle 110 is shown defining a pencil-grip configuration, other suitable handle configurations are also contemplated such as, for example, a pistol-grip, a plunger-grip, etc.

Elongated body 120 extends distally from handle 110 and defines sufficient length to enable a distal end portion of elongated body 120 to be positioned adjacent an internal surgical site, e.g., within a vertebral body, while handle 110 remains externally disposed. Elongated body 120 may include one or more sections that are straight, pre-bent, rigid, flexible, malleable, and/or articulatable. For example, elongated body 120 may define an at least partially flexible configuration wherein a distal section 122 thereof is biased towards a curved configuration, as illustrated in FIG. 1, although other configurations are also contemplated. Elongated body 120 may be resiliently flexible from this curved configuration to a substantially linear configuration to enable insertion of elongated body through introducer 200. In such configurations, upon emergence of elongated body 120 from a distal end of introducer 200, e.g., within the internal surgical site, the portion of elongated body 120 that extends distally from introducer 200 is thus permitted to resiliently return towards the curved configuration. Elongated body 120 (or a portion thereof) may be formed from nitinol or other suitable shape memory material.

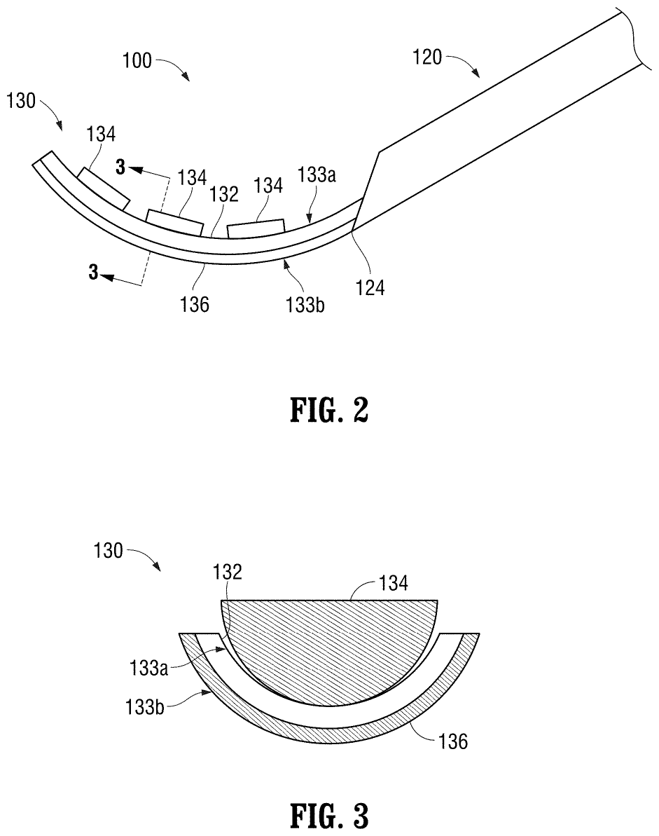

With additional reference to FIG. 2, elongated body 120 may define a sharpened distal tip 124 to facilitate penetration through tissue such as bone or may define a blunt configuration. End effector assembly 130 is selectively deployable relative to elongated body 120 from a retracted position (FIG. 1), wherein end effector assembly 130 is substantially disposed within elongated body 120, to a deployed position (FIG. 2), wherein end effector assembly 130 extends distally from elongated body 120. End effector assembly 130 may be deployed and retracted via a slider 152 of user interface 150, although other suitable actuators, e.g., triggers, plungers, wheels, etc., are also contemplated. End effector assembly 130 and user interface 150 are described in greater detail below.

Referring again to FIG. 1, introducer 200 includes a proximal hub 210 and a distal sheath 220 extending distally from proximal hub 210. Distal sheath 220 includes a tissue-penetrating tip 222 to facilitate insertion of introducer 200 through tissue, e.g., bone, into an internal surgical site. A longitudinal lumen 230 extends through proximal hub 210 and distal sheath 220 to enable insertion of elongated body 120 of ablation device 100 through introducer 200, distally therefrom, and into the internal surgical site. Distal sheath 220 may include one or more sections that are straight, pre-bent, rigid, flexible, malleable, and/or articulatable. As noted above, in some aspects, introducer 200 is omitted.

Generator 300 is configured to provide suitable energy to ablation device 100 for treating, e.g., ablating, tissue therewith. For example, generator 300 may provide Radio Frequency (RF) energy to ablation device 100 for ablating tissue, as detailed below, although other suitable forms of energy, e.g., microwave, ultrasonic, thermal, etc., and/or tissue treatments, are also contemplated.

With reference to FIGS. 2 and 3, in conjunction with FIG. 1, end effector assembly 130 of ablation device 100 includes a base electrode, e.g., tongue 132, a plurality of electrodes 134 disposed on a first face 133a of tongue 132, and an insulating layer 136 disposed on a second, opposite face 133b of tongue 132. Tongue 132 may be resiliently flexible to enable flexion of tongue 132 between a curved configuration, wherein tongue 132 defines a curvature along at least a portion of a longitudinal length thereof, and a linear configuration, wherein tongue 132 extends substantially linearly along the longitudinal length thereof. The linear configuration of tongue 132 may correspond to the retracted position of end effector assembly 130. More specifically, with tongue 132 disposed in the linear configuration, end effector assembly 130 is capable of being received within elongated body 120 to define the retracted position (FIG. 1). End effector assembly 130 is deployable from this retracted position to the deployed position (FIG. 2), wherein end effector assembly 130 extends distally from elongated body 120 and is permitted to move to the curved configuration. In some aspects, tongue 132 extends radially outwardly beyond the outer radial dimension of elongated body 120 in the curved configuration. Tongue 132 may be biased towards the curved configuration such that, upon deployment of end effector assembly 130 from elongated body 120, tongue 132 resiliently returns towards the curved configuration. In the curved configuration, first face 133a of tongue 132 (including electrodes 134) is disposed on the inside portion of the curve while second face 133b of tongue 132 (including insulating layer 136) is disposed on the outside portion of the curve. Tongue 132 may define a radiused curvature in the curved configuration. Alternatively, tongue 132 may define another curvature, multiple curvatures, straight portions, angled portions, combinations thereof, etc. in the curved configuration. Tongue 132 (or a portion thereof) may be formed from nitinol or other suitable shape memory material to enable flexion of tongue 132 between the linear and curved configurations.

As illustrated in FIG. 3, tongue 132 defines a radiused transverse cross-sectional configuration although U-shaped or V-shaped configurations are also contemplated. Tongue 132, more specifically, in aspects, extends from about 45 degrees to about 135 degrees about the circumference of a circle in transverse cross-section; in other aspects, from about 60 degrees to about 120 degrees about the circumference of a circle in transverse cross-section; or in still other aspects, defines a semi-circle in transverse cross-sectional configuration (extending approximately 90 degrees about the circumference of a circle). First face 133a of tongue 132 (including electrodes 134) is disposed on the concave side (in cross-section) of tongue 132 while second face 133b of tongue 132 (including insulating layer 136) is disposed on the convex side (in cross-section) of tongue 132.

Referring again to FIGS. 2 and 3, in aspects, tongue 132 (or a portion thereof) is formed from an electrically-conductive material adapted to connect to generator 300 (e.g., via a lead wire (not shown) extending from end effector assembly 130 through elongated body 120, handle 110, and cable 160 to generator 300). In this manner, tongue 132 may function as a return electrode in a bipolar or monopolar RF circuit while electrodes 134 function as the active electrodes, as detailed below.

Electrodes 134 are disposed on first face 133a of tongue 132 and longitudinally spaced-apart from one another along at least a portion of the longitudinal length of tongue 132. Although three electrodes 134 are shown, any suitable number of electrodes e.g., two electrodes 134 or more than three electrodes 134, are also contemplated. Electrodes 134 are formed from an electrically-conductive material adapted to connect to generator 300 (e.g., via lead wires (not shown) extending from end effector assembly 130 through elongated body 120, handle 110, and cable 160 to generator 300). Electrodes 134 may be collectively connected to generator 300 (e.g., via a common lead wire) such that electrodes 134 are energizable together, may be independently connected to generator 300 (e.g., via separate lead wires) such that electrodes 134 are independently energizable, or may be connected to generator 300 in one or more groups (e.g., via one or more group lead wires) such that electrodes 134 in a group are energizable together while different groups of electrodes 134 may be energized independently. Electrodes 134 are electrically-insulated from tongue 132, e.g., via an insulative layer (not shown) disposed therebetween.

In aspects, one or more electrodes 134 may function as the active electrode(s) in a bipolar RF circuit while tongue 132 functions as the return electrode. Thus, energy may be conducted between the electrode(s) 134 (charged to a first potential, e.g., a positive “+” potential) and tongue 132 (charged to a second, different potential, e.g., a negative “−” potential) and through tissue to treat, e.g., ablate tissue. Alternatively, one or more electrodes 134 (and/or tongue 132) may be charged to the first potential while one or more other electrodes 134 (and/or tongue 132) is charged to the second potential (or a third potential) to establish a bipolar RF circuit wherein energy may be conducted between the electrode(s) 134 (and/or tongue 132) and the other electrode(s) 134 (and/or tongue 132) and through tissue to treat, e.g., ablate tissue. Further still, multipolar configurations are also contemplated such as, for example, a three-phase RF circuit wherein at least three potentials are utilized between the electrodes 134 and/or tongue 132 to enable conduction of energy between any two electrodes 134 energized to different potentials and/or between tongue 132 and any electrode 134 energized to a different potential from tongue 132 and through tissue to treat, e.g., ablate tissue. In aspects, some electrodes 134 and/or tongue 132 may be turned “OFF” in some modes of operation, and/or may be energized sequentially, in alternating fashion, or according to any other suitable pattern. Monopolar RF circuits are also contemplated. Regardless of the particular energization of electrodes 134 and tongue 132, the energization may be continuous, pulsed, combinations thereof, or in any other suitable manner. Likewise, the intensity and duration of energization may be controlled to achieve a desired tissue treatment, e.g., ablation, while inhibiting collateral damage. Selecting a mode of activation, adjusting activation settings, and/or initiating activation of end effector assembly 130 may be performed via buttons 154 of user interface 150 (FIG. 1) and/or via generator 300 (FIG. 1), as detailed below.

With momentary reference to FIG. 3, electrodes 134 may define semi-cylindrical configurations (semi-circular transverse cross-sectional configurations); may define a partial cylindrical configuration extending from about 45 degrees to about 135 degrees about the circumference of a cylinder (of a circle in transverse cross-section); or may define a partial cylindrical configuration extending from about 60 degrees to about 120 degrees about the circumference of a cylinder (of a circle in transverse cross-section). U-shaped, V-shaped, or other configurations are also contemplated. Electrodes 134 may be shaped complementary to first face 133a of tongue 132 or differently therefrom. Electrodes 134 may protrude beyond the outer dimensions of tongue 132, e.g., in the facing direction of first face 133a, may extend to the outer dimensions of tongue 132, e.g., in the facing direction of first face 133a, or may be recessed within the outer dimensions of tongue 132. The electrodes 134 may be similar to one another or different from one another, e.g., defining different lengths, heights, etc.

Referring again to FIGS. 2 and 3, insulating layer 136 is formed from an electrically and thermally insulating material and, as noted above, is disposed on second face 133b of tongue 132, on the opposite side of tongue 132 as compared to electrodes 134. In this manner, insulating layer 136 inhibits the conduction of thermal and electrical energy outwardly from second face 133b of tongue 132. Insulating layer 136 may be coated, adhered, overmolded, or otherwise disposed on second face 133b of tongue 132

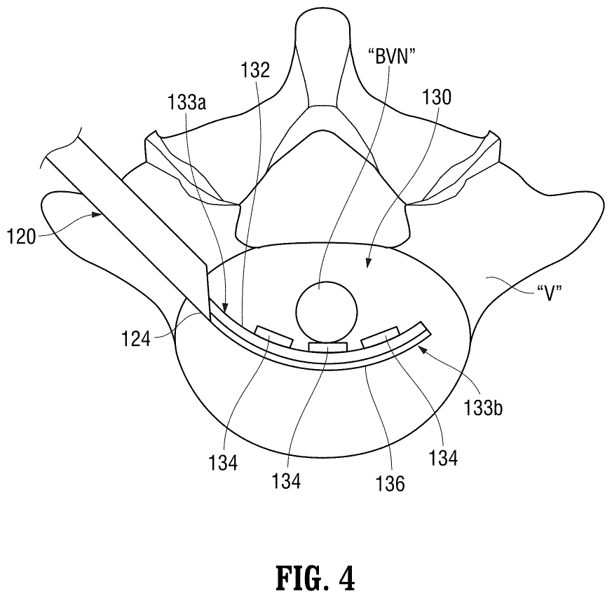

With reference to FIG. 4, in use, with end effector assembly 130 disposed in the retracted position, elongated body 120 is inserted into the vertebral body “V” to a position adjacent a basivertebral nerve “BVN,” e.g., adjacent the cancellous bone containing the basivertebral nerve “BVN.” Elongated body 120 may penetrate the vertebral body “V” itself or may be utilized in conjunction with introducer 200 (FIG. 1) to facilitate positioning distal tip 124 of elongated body 120 within the vertebral body “V” and adjacent the basivertebral nerve “BVN,” anteriorly thereof. Once this position has been achieved, end effector assembly 130 is moved to the deployed position such that tongue 132 is positioned anteriorly of and extends at least partially about the basivertebral nerve “BVN” with first face 133a thereof (and, thus, electrodes 134) facing posteriorly and towards the basivertebral nerve “BVN,” while second face 133b of tongue faces anteriorly and away from the basivertebral nerve “BVN.”

The curved configuration of tongue 132 along at least a portion of its length enables tongue 132 to partially circumferentially surround the basivertebral nerve “BVN.” In this manner, with end effector assembly 130 disposed in the deployed position, electrodes 134 are substantially oriented radially inwardly towards the basivertebral nerve “BVN.” Once this position has been achieved, electrodes 134 and/or tongue 132 may be energized in any suitable arrangement, pattern, etc., to conduct energy therebetween and through the cancellous bone containing the basivertebral nerve “BVN” to heat and thereby treat, e.g., ablate, the basivertebral nerve “BVN.” In aspects, the basivertebral nerve “BVN” is treated to achieve at least partial denervation, e.g., by ablating at least some of the nerve fibers associated with the basivertebral nerve “BVN.” As the basivertebral nerve “BVN” is heated and, thus, treated, insulating layer 136 protects surrounding tissue structures by confining the thermal and electrical energy from end effector assembly 130 to the posterior direction, e.g., towards the basivertebral nerve “BVN.”

In aspects, end effector assembly 130 may be repositioned, e.g., by advancing or retracting elongated body 120, rotating elongated body 120, further deploying end effector assembly 130, or partially retracting end effector assembly 130, to enable further treatment of the basivertebral nerve “BVN” from a different orientation and/or position. Once treatment is complete, end effector assembly 130 may be returned to the retracted position (FIG. 1) and ablation device 100 removed from the vertebral body “V.”

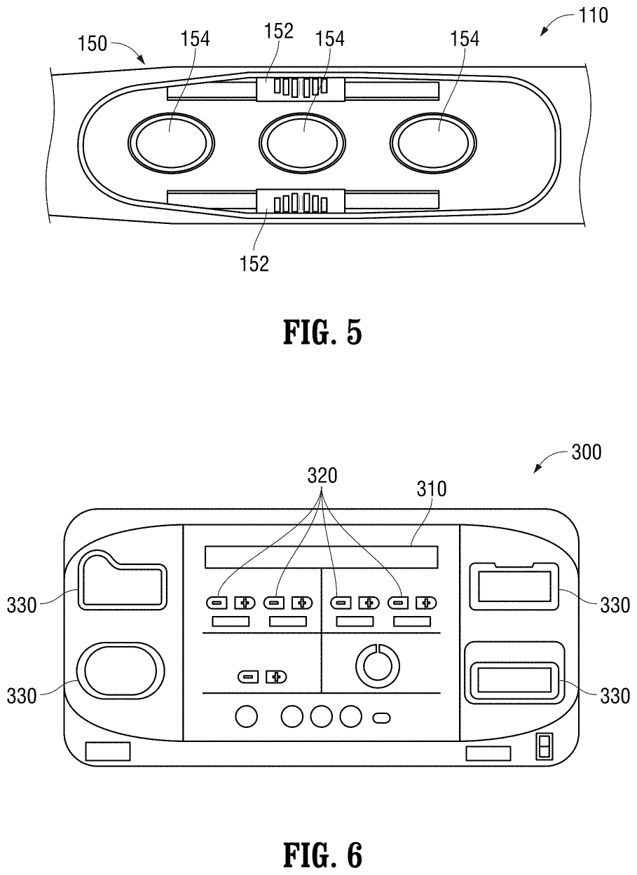

Turning to FIG. 5, in conjunction with FIG. 2, user interface 150 of handle 110 of ablation device 100 is shown. User interface 150 enables control of end effector assembly 130, e.g., deployment, setting adjustment, and/or activation of end effector assembly 130. User interface 150 includes a pair of sliders 152 and a plurality of buttons 154, although alternative or additional user interface components are also contemplated such as, for example, rocker switches, dials, touch-screen graphical user interfaces (GUI), etc. One or both of sliders 152 may be selectively slidable to deploy and retract end effector assembly 130, e.g., wherein distal sliding deploys end effector assembly 130 while proximal sliding retracts end effector assembly 130. In aspects, sliders 152 are associated with an underlying safety switch (not shown) that inhibits activation when end effector assembly 130 is disposed in the retracted position.

The buttons 154 may include multi-click buttons wherein the user depresses a button 154 one or more times to make a desired selection; multi-stage buttons (continuous or step-wise) wherein the user depresses a button 154 to a position corresponding to the desired selection; ON-OFF buttons wherein the user depresses and holds a button 154 to maintain an ON condition; and/or other suitable buttons. One or more of buttons 154 may be configured to enable mode selection between various modes of operation. The modes of operation may include, for example: a first mode wherein energy is conducted between the electrodes 134 and tongue 132; a second mode wherein energy is conducted between the electrodes 134 themselves (and/or tongue 132); and/or one or more third modes of operation wherein energy is conducted between select electrodes 134 and other electrodes and/or tongue 132. One or more of the buttons 154 may alternatively or additionally be configured, for example, to enable selection of an energy intensity, e.g., LOW power or HIGH power; an energy duration setting, e.g., 1 minute, 2 minutes, or 5 minutes; or an energy profile, e.g., continuous, pulsed, etc. One of the buttons 154 is configured as an activation button, e.g., to initiate and terminate the supply of energy to end effector assembly 130. The mode of operation, energy intensity, energy profile, and/or energy duration may be selected, for example, based on the needs of the procedure, patient anatomy, placement of the end effector assembly 130, etc. Pre-set programs including select modes and energy settings are also contemplated and may likewise be activated by one or more of buttons 154.

Regardless of the particular configuration of user interface 150, user interface 150 communicates with generator 300 (FIG. 1), e.g., via one or more lead wires (not shown) extending through cable 160 (FIG. 1) to enable selective activation of end effector assembly 130 and implementation of the selected activation settings.

With reference to FIG. 6, in conjunction with FIGS. 1 and 2, generator 300 includes a display 310, a plurality of user interface features 320, e.g., buttons, touch-screen GUIs, switches, etc., and one or more plug ports 330. Display 310 is configured to display operating parameters, settings, alerts, and/or other information associated with use of ablation device 100. User interface features 320 enable control of end effector assembly 130, e.g., deployment, setting adjustment, and/or activation of end effector assembly 130, in addition to or as an alternative to user interface 150 of ablation device 100. One of the plug ports 330 is configured to receive a plug (not shown) associated with cable 160 to enable electrical coupling of ablation device 100 with generator 300. Where additional plug ports 330 are provided, such plug ports 330 may enable connection of auxiliary device(s) and/or other energy-based device(s) to generator 300.

Generator 300 is configured to produce electrosurgical energy, e.g., RF monopolar or bipolar energy, for output to end effector assembly 130 of ablation device 100 for treating, e.g., ablating tissue therewith. Generator 300 may further include feedback circuitry configured to monitor voltage, current, impedance, temperature, etc., and to control energy output based thereon to implement the selected activation settings.

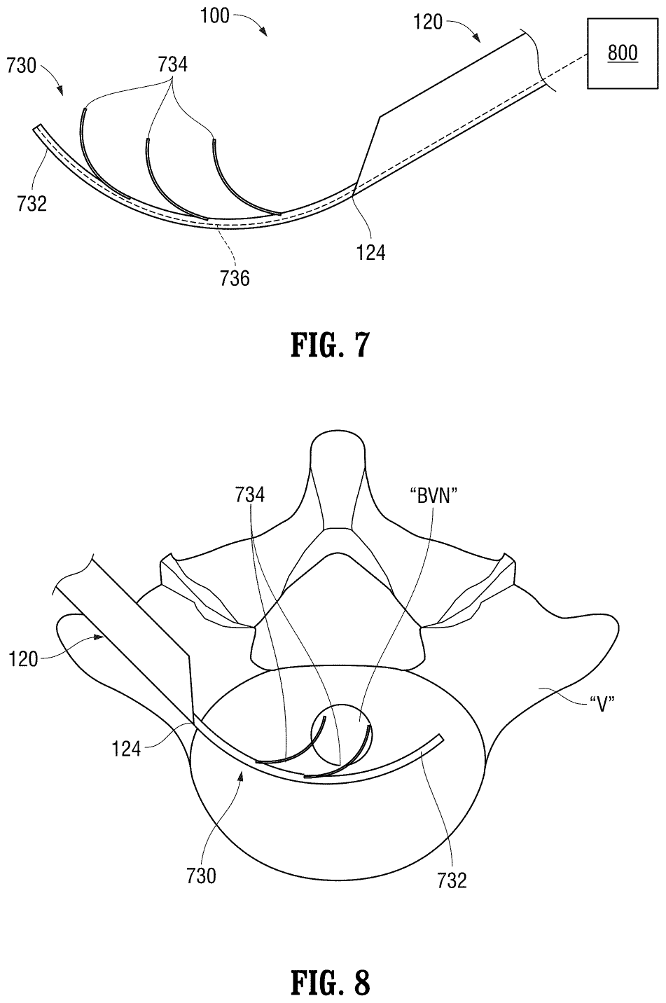

Referring to FIG. 7, ablation device 100 is shown in a deployed position including another end effector assembly 730 in accordance with the present disclosure. Except as explicitly contradicted below, the features of ablation device 100 detailed above with respect to end effector assembly 130 (FIGS. 2-4) are equally applicable for use with end effector assembly 730 and, thus, may not be repeated hereinbelow for purposes of brevity.

End effector assembly 730 is selectively deployable relative to elongated body 120 from a retracted position (FIG. 1), wherein end effector assembly 730 is substantially disposed within elongated body 120, to a deployed position (FIG. 7), wherein end effector assembly 730 extends distally from elongated body 120. End effector assembly 730 may be deployed and retracted via a slider 152 of user interface 150 (FIGS. 1 and 5), or any other suitable deployment mechanism. End effector assembly 730 includes a base electrode, e.g., shaft 732, and a plurality of electrode tines 734 extending from or extendable from shaft 732.

Shaft 732 may define a circular cross-sectional configuration, a polygonal cross-sectional configuration, or any other suitable cross-sectional configuration and may be resiliently flexible to enable flexion of shaft 732 between a curved configuration, wherein shaft 732 defines a curvature along at least a portion of a longitudinal length thereof, and a linear configuration, wherein shaft 732 extends substantially linearly along the longitudinal length thereof. The linear configuration of shaft 732 may correspond to the retracted position of end effector assembly 730. In the deployed position of end effector assembly 730, with elongated body 120 no longer constraining shaft 732, shaft 732 is permitted to move, e.g., under resilient bias, towards the curved configuration. In the curved configuration, electrode tines 734 extend from or are extendable from the inside portion of the curve defined along the length of shaft 732. Shaft 732 (or a portion thereof) may be formed from nitinol or other suitable shape memory material to enable flexion thereof between the linear and curved configurations.

Shaft 732 (or a portion thereof) is formed from an electrically-conductive material adapted to connect to generator 300 (e.g., via a lead wire (not shown) extending from end effector assembly 730 through elongated body 120, handle 110, and cable 160 to generator 300 (see FIG. 1)). In this manner, shaft 732 may function as a return electrode in a monopolar RF circuit while electrode tines 734 function as the active electrodes, as detailed below.

Electrode tines 734, as noted above, may extend from or may be extendable from shaft 732. That is, the fixed ends of electrode tines 734 may be fixed relative to shaft 732 with electrode tines 734 extending therefrom to the free ends thereof. In such configurations, electrode tines 734 are resiliently flexible to enable electrode tines 734 to be collapsed inwardly towards shaft 732 in the retracted position of end effector assembly 730 and to resiliently return to extend outwardly from shaft 732 in the deployed position of end effector assembly 730. Alternatively, electrode tines 734 may be selectively extendable through tine ports defined within shaft 732. More specifically, the fixed ends of electrode tines 734 may be coupled to a drive structure (not shown) associated within one of the sliders 152 of user interface 150 (see FIG. 1) such that electrode tines 734 may be selectively deployed from shaft 732, together with the deployment of end effector assembly 730 from elongated body 120 or independently thereof. Any suitable number of electrode tines 734 may be provided.

Electrode tines 734 are spaced-apart from one another along at least a portion of a length of shaft 732 and are disposed on the same side of shaft 732, e.g., the inside portion thereof. Electrode tines 734 may include tissue-penetrating free ends to facilitate penetration of electrode tines 734 through tissue. Electrode tines 734 may define arcuate, angled, or other suitable configurations. In aspects where electrode tines 734 are arcuate, each electrode tine 734 may be curved in a similar direction, although other configurations are also contemplated. In aspects, electrode tines 734 are curved in a similar direction as shaft 732 in the curved configuration thereof.

Electrode tines 734 are formed from an electrically-conductive material adapted to connect to generator 300 (e.g., via lead wires (not shown) extending from end effector assembly 730 through elongated body 120, handle 110, and cable 160 to generator 300) (see FIG. 1). Electrode tines 734 may be collectively connected to generator 300 (FIG. 1) (e.g., via a common lead wire) such that electrode tines 734 are energizable together, may be independently connected to generator 300 (FIG. 1) (e.g., via separate lead wires) such that electrode tines 734 are independently energizable, or may be connected to generator 300 (FIG. 1) in one or more groups (e.g., via one or more group lead wires) such that electrode tines 734 in a group are energizable together while different groups of electrode tines 734 may be energized independently. Electrode tines 734 are electrically-insulated from shaft 732, e.g., via suitable insulation (not shown).

In aspects, one or more electrode tines 734 may function as the active electrode(s) in a monopolar RF circuit while shaft 732 functions as the return electrode in the monopolar circuit. Thus, energy may be conducted between the electrode tine(s) 734 (charged to a first potential, e.g., a positive “+” potential) and shaft 732 (charged to a second, different potential, e.g., a negative “−” potential) and through tissue to treat, e.g., ablate tissue. Alternatively, one or more electrode tines 734 (and/or shaft 732) may be charged to the first potential while one or more other electrode tines 734 (and/or shaft 732) is charged to the second potential (or a third potential) to establish a bipolar RF circuit wherein energy may be conducted between the electrode tines 734 (and/or shaft 732) and the other electrode tines 734 (and/or shaft 732) and through tissue to treat, e.g., ablate tissue. Further still, multipolar configurations are also contemplated such as, for example, a three-phase RF circuit wherein at least three potentials are utilized between the electrode tines 734 and/or shaft 732 to enable conduction of energy between any two electrode tines 734 energized to different potentials and/or between shaft 732 and any electrode tines 734 energized to a different potential from shaft 732 and through tissue to treat, e.g., ablate tissue. In aspects, some electrode tines 734 and/or shaft 732 may be turned “OFF” in some modes of operation, and/or may be energized sequentially, in alternating fashion, or according to any other suitable pattern.

In aspects, end effector assembly 730 further includes one or more cooling lumens 736 extending through shaft 732 and coupled to a fluid source 800 (including a pump) to enable circulation of cooling fluid through shaft 732 via cooling lumen(s) 736. The one or more cooling lumens 736 may be positioned on the inside portion of shaft 732 (in the curved configuration thereof), the outside portion thereof, on both the inside and outside portions, and/or may be centrally located.

With additional reference to FIG. 8, in use, with end effector assembly 730 disposed in the retracted position, elongated body 120 is inserted into the vertebral body “V” to a position adjacent a basivertebral nerve “BVN,” e.g., adjacent the cancellous bone containing the basivertebral nerve “BVN.” Elongated body 120 may penetrate the vertebral body “V” itself or may be utilized in conjunction with introducer 200 (FIG. 1) to facilitate positioning distal tip 124 of elongated body 120 within the vertebral body “V” and adjacent the basivertebral nerve “BVN,” anteriorly thereof. Once this position has been achieved, end effector assembly 730 is moved to the deployed position such that shaft 732 is positioned anteriorly of and extends at least partially about the basivertebral nerve “BVN” with the inside portion thereof facing posteriorly and towards the basivertebral nerve “BVN.”

If not already done so, electrode tines 734 are then extended from shaft 732 in a posterior direction, e.g., from the inside portion of shaft 732, such that the free ends of electrode tines 734 extend into the basivertebral nerve “BVN.” Thereafter, electrode tines 734 and/or shaft 732 may be energized in any suitable arrangement, pattern, etc., to conduct energy therebetween and through the cancellous bone containing the basivertebral nerve “BVN” to heat and thereby treat, e.g., ablate, the basivertebral nerve “BVN.” In aspects, the basivertebral nerve “BVN” is treated to achieve at least partial denervation, e.g., by ablating at least some of the nerve fibers associated with the basivertebral nerve “BVN.” Cooling, e.g., via circulating cooling fluid through shaft 732, may be initiated automatically upon energy activation, intermittently during energy activation, independently (user-controlled), or in any other suitable manner. As the basivertebral nerve “BVN” is heated, the cooling of shaft 732 protects surrounding tissue structures by confining the thermal and electrical energy from end effector assembly 730 to the posterior direction, e.g., towards the basivertebral nerve “BVN.” In aspects, the cooling fluid circulates on the inside portion (posterior side) of shaft 732 to facilitate energy conduction in the posterior direction, although other configurations are also contemplated.

As an alternative to cooling via circulation of cooling fluid, other cooling mechanisms may be employed such as, for example, passive coolers (heat sinks, heat pipes, fins, etc.) or active coolers (Peltier (thermoelectric) coolers, etc.).

In aspects, end effector assembly 730 and, more specifically, electrode tines 734 thereof, may be repositioned for subsequent treatment of the basivertebral nerve “BVN” from a different position and/or orientation. Once treatment is complete, end effector assembly 130 may be returned to the retracted position (FIG. 1) and ablation device 100 removed from the vertebral body “V.”

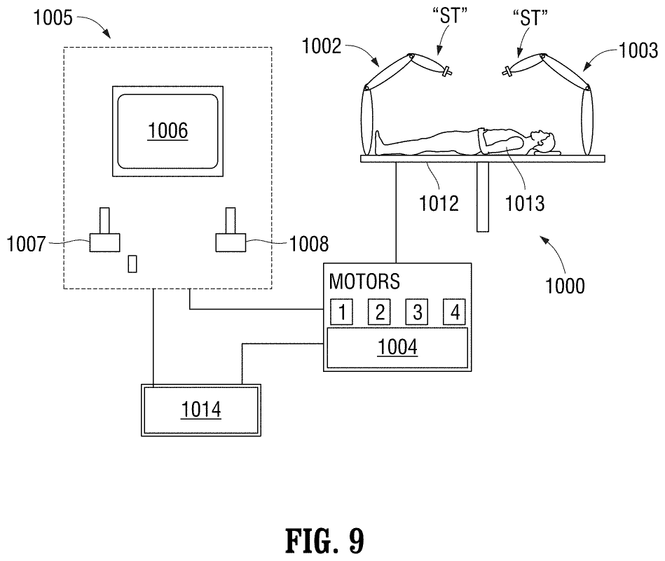

Turning to FIG. 9, a robotic surgical system 1000 configured for use in accordance with the present disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, any of the ablation devices of the present disclosure, the introducer of the present disclosure, an endoscope or other visualization device, etc. More specifically, with respect to the ablation devices detailed herein, the user-activation and actuation components are replaced with robotic inputs to enable a robot to provide the desired activation(s) and actuation(s) similarly as detailed above. That is, in robotic implementations, the ablation devices function similarly according to any of the aspects above except that the ablation devices are directly manipulated, activated, and/or actuated by a robot arm 1002, 1003 rather than a human surgeon.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

While several configurations of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. An ablation device, comprising:
a handle;
an elongated body extending distally from the handle; and
an end effector assembly selectively deployable relative to the elongated body, the end effector assembly including:
a shaft having a curved configuration along at least a portion of a length thereof defining an inside portion and an outside portion, the shaft adapted to connect to a source of Radio Frequency (RF) energy; and
a plurality of electrode tines spaced-apart along the shaft and extending from the inside portion thereof, each electrode tine of the plurality of electrode tines adapted to connect to the source RF energy,
wherein at least one electrode tine of the plurality of electrode tines and the shaft are configured to conduct RF energy therebetween and through tissue to treat tissue.

2. The ablation device according to claim 1, wherein each electrode tine of the plurality of electrode tines includes a tissue-penetrating tip.

3. The ablation device according to claim 1, wherein at least two electrode tines of the plurality of electrode tines are configured to conduct RF energy therebetween.

4. The ablation device according to claim 1, wherein the plurality of electrode tines is selectively extendable relative to the shaft.

5. The ablation device according to claim 1, wherein each electrode tine of the plurality of electrode tines defines a curved configuration and is curved in a similar direction as the shaft.

6. The ablation device according to claim 1, further comprising at least one cooling lumen extending through the shaft, the at least one cooling lumen configured to receive cooling fluid to cool at least a portion of the shaft.

7. The ablation device according to claim 1, wherein the shaft is resiliently biased towards the curved configuration and is resiliently flexible from a substantially linear configuration, corresponding to a retracted position of the end effector assembly relative to the elongated body, to the curved configuration, corresponding to a deployed position of the end effector assembly relative to the elongated body.

8. An ablation device, comprising:
a handle;
an elongated body extending distally from the handle; and
an end effector assembly selectively deployable relative to the elongated body, the end effector assembly including:
a tongue having a curved configuration along at least a portion of a length thereof defining an inside portion and an outside portion, the tongue defining a curved transverse cross-sectional configuration defining a concave side and a convex side, the concave side corresponding to the inside portion and the convex side corresponding to the outside portion, the tongue adapted to connect to a source of Radio Frequency (RF) energy;
a plurality of electrodes spaced-apart along the tongue and disposed on the concave side of the tongue, each electrode of the plurality of electrodes adapted to connect to the source RF energy; and
an insulating layer disposed on the convex side of the tongue,
wherein at least one electrode of the plurality of electrodes and the tongue are configured to conduct RF energy therebetween and through tissue to treat tissue.

9. The ablation device according to claim 8, wherein at least two electrodes of the plurality of electrodes are configured to conduct RF energy therebetween.

10. The ablation device according to claim 8, wherein the tongue is resiliently biased towards the curved configuration and is resiliently flexible from a substantially linear configuration, corresponding to a retracted position of the end effector assembly relative to the elongated body, to the curved configuration, corresponding to a deployed position of the end effector assembly relative to the elongated body.

11. The ablation device according to claim 8, further comprising a user interface disposed on the handle, the user interface including a first control for selectively deploying the end effector assembly relative to the elongated body and a second control for selectively activating the supply of energy to the at least one electrode and the tongue.

12. The ablation device according to claim 8, wherein the elongated body is resiliently flexible.

13. The ablation device according to claim 8, wherein the elongated body defines a tissue-penetrating tip.

14. A method of ablating tissue, comprising:
inserting an elongated body into a vertebral body of a patient anteriorly of a basivertebral nerve;
deploying an end effector assembly from the elongated body such that a base electrode of the end effector assembly is positioned anteriorly of a basivertebral nerve with a plurality of electrodes of the end effector assembly facing posteriority towards the basivertebral nerve; and
conducting RF energy between at least one electrode of the plurality of electrodes and the base electrode causing RF energy to be conducted through cancellous bone containing the basivertebral nerve to ablate the basivertebral nerve.

15. The method according to claim 14, wherein deploying the end effector assembly orients the plurality of electrodes substantially radially inwardly towards the basivertebral nerve.

16. The method according to claim 14, wherein deploying the end effector assembly includes penetrating the basivertebral nerve with at least one electrode of the plurality of electrodes.

17. The method according to claim 14, wherein deploying the end effector assembly orients an insulating layer disposed on the base electrode on an anterior side of the base electrode to inhibit energy conduction anteriorly.

18. The method according to claim 14, further comprising circulating cooling fluid through a portion of the base electrode to promote RF energy conduction posteriorly.

19. The method according to claim 14, wherein conducting RF energy further includes conducting RF energy between at least two electrodes of the plurality of electrodes.
